(19) United States
(12) Patent Application Publication
(10) Pub. No.: US 2015/0336879 A1
(43) Pub. Date: Nov. 26, 2015

(54) PROCESS FOR THE SYNTHESIS OF OMEGA-UNSATURATED NITRILE-ACID/ESTER IN WHICH TWO TYPES OF CROSS METATHESIS ARE ALTERNATED CONSECUTIVELY, SWING PROCESS

(71) Applicant: ARKEMA FRANCE, Colombes (FR)

(72) Inventors: Jean-Luc DUBOIS, Millery (FR); Jean-Luc COUTURIER, Lyon (FR)

(73) Assignee: ARKEMA FRANCE, Colombes (FR)

(21) Appl. No.: 14/759,573

(22) PCT Filed: Jan. 7, 2013
(86) PCT No.: PCT/IB2013/000017

§ 371 Date: Jul. 7, 2015

(51) Int. Cl. C07C 253/30; C08G 69/00; C07C 227/04

(52) U.S. Cl. CPC C07C 253/30; C07C 227/04; C08G 69/00

(57) ABSTRACT

A process for the synthesis of a monounsaturated nitrile-ester (acid) by cross metathesis starting from an unsaturated compound I including at least one trivalent nitrile, acid or ester functional group, wherein the process includes a stage in which two types of cross metathesis are alternated consecutively: — a first type of cross metathesis cm1 with an acrylic compound II chosen from acrylonitrile, acrylic acid or an acrylic ester, one of the compounds I or II including a nitrile functional group and the other of these compounds II or I including an acid or ester functional group, and—a second type of cross metathesis cm2 with a light C₂ to C₄ alkene compound III. Also, a process for the synthesis of an α,ω-amino ester (acid) obtained by a process including a stage in which the two types of cross metathesis defined above are alternated consecutively, followed by a hydrogenation stage.

The work which led to this invention received financial support from the European Union in the context of Framework Programme 7 (FP7/2007-2013) under the project number No. 241718 EUROBIOREF.

### FIELD OF THE INVENTION

The invention relates to an improved process for the synthesis of ω-unsaturated nitrile-acid or nitrile-ester by metathesis starting from unsaturated fatty acids or esters.

ω-Unsaturated nitrile-acid or nitrile-ester compounds are used in particular for the manufacture of long-chain α,ω-aminoalkanoic acids or esters, that is to say for which the chain comprises at least 8 carbon atoms, themselves used in the specialty polymers industry, in particular the high-performance polyamides industry.

### TECHNICAL BACKGROUND

The polyamides industry uses a whole range of monomers formed from diamines and diacids, from lactams and in particular from ω-amino acids. The latter are defined by the length of methylene chain (—CH₂)ₙ— separating two amide functional groups —CO—NH—. These monomers are generally manufactured by a chemical synthesis route using, as starting materials, C₂ to C₄ olefins, cycloalkanes or benzene, which hydrocarbons result from fossil sources. For example, C₂ olefins are used to manufacture the C₉ amino acid used in nonanoic acid; C₄ olefins are used to manufacture hexamethylenediamine; laurolactam and caprolactam are manufactured from cycloalkanes; adipic acid, Nylon 6 and Nylon 6,6 are manufactured from benzene.

Current developments with regard to the environment are resulting in the use of natural starting materials originating from a renewable source being favored in the fields of energy and chemistry. This is the reason why some studies have been taken up to develop, industrially, processes using fatty acids/esters as starting material in the manufacture of these monomers.

This type of approach has only a few industrial examples. One of the rare examples of an industrial process using a natural fatty acid as starting material is that of the manufacture, from the ricinoleic acid extracted from castor oil, of 11-aminoundecanoic acid, which forms the basis of the synthesis of Rilsan® 11. This process is described in the work “Les Procédés de Pétrochimie” [Petrochemical Processes] by A. Chauvel et al., which appeared in Editions Technip (1986). 11-Aminoundecanoic acid is obtained in several stages. The first consists of a methanolysis of castor oil in a basic medium, producing methyl ricinoleate, which is subsequently subjected to a pyrolysis in order to obtain, on the one hand, heptanaldehyde and, on the other hand, methyl undecylenate. The latter is converted to the acid form by hydrolysis. Subsequently, the acid formed is subjected to a hydrobromination to give the ω-brominated acid, which is converted by ammoniation to 11-aminoundecanoic acid.

In this “bioroute”, the main studies have related to the synthesis of 9-aminononanoic acid, which is the precursor of Nylon 9, from oleic acid of natural origin.

For its part, the Applicant Company has carried out studies in this field. It has described, in the patent document FR2912741, a process for the synthesis of a whole range of amino acid/ester of this type from a natural long-chain fatty acid/ester by subjecting the latter to a cross catalytic metathesis reaction with an unsaturated compound comprising a nitrile functional group, followed by hydrogenation. It is also described, in the patent document FR2938533, a process for the synthesis of ω-aminoalkanoic acids or of their esters from natural long-chain unsaturated fatty acids passing through an intermediate compound of ω-unsaturated nitrile type, one of the alternative forms of which employs, in the final phase, a cross metathesis of the ω-unsaturated nitrile with a compound of acrylate type. Finally, it is described, in the patent document FR2941694, an alternative form of the preceding process in which the intermediate compound is of the unsaturated dinitrile type. These processes result, on conclusion of a stage of hydrogenation of the nitrile functional group and of the double bond, in the manufacture of amino acids. In the patent document FR2959742, the process described has the object of improving the performances of the processes successively employing a cross metathesis and a hydrogenation.

In these processes of the prior art, the cross metathesis reactions, generally carried out between an ω-unsaturated fatty nitrile and an acrylate or between an ω-unsaturated fatty ester and acrylonitrile, result not only in the desired product, which is a nitrile-ester, but also in products resulting from a homometathesis reaction of the fatty substances, such as dinitriles and diesters respectively. It is possible, by increasing the amounts of catalyst employed, the reaction times and/or the ratios between the reactants, to convert these coproducts resulting from the homometathesis into nitrile-ester; however, these solutions prove to be expensive and not very productive.

Moreover, the products of the homometathesis reactions are heavy long-chain diester or dinitrile products which it is difficult to separate from the reaction medium and to purify. The applications of this type of product are limited and very often dissociated from the industrial applications desired for nitrile-esters.

There thus exists a real need to develop a process for the synthesis of nitrile-ester/acid in which the amount of coproducts resulting from the homometathesis reactions is reduced.

The Applicant Company has found that, surprisingly, it is possible to minimize the amounts of fatty diester or dinitrile coproducts resulting from homometathesis reactions by carrying out various alternated consecutively cross metathesis reactions.

### DETAILED DESCRIPTION OF THE INVENTION

In the present description, it is specified that, when reference is made to intervals, the expressions of the type “ranging from . . . to” or “comprising from . . . to” include the limits of the interval. Conversely, the expressions of the type “between . . . and . . . ” exclude the limits of the interval.

Unless otherwise mentioned, the percentages expressed are molar percentages. Unless otherwise mentioned, the parameters to which reference is made are measured at atmospheric pressure.

A subject matter of the present invention is thus a process for the synthesis, by cross metathesis, of a long-chain nitrile-acid or nitrile-ester with an improved yield.

The term “cross metathesis” is understood to mean, within the meaning of the invention, the following general reaction scheme:

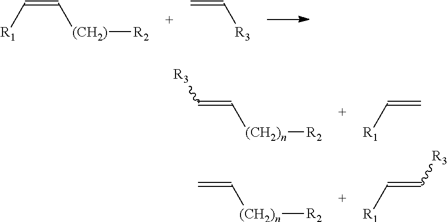

R₁=H, a C₁ to C₁₁ alkyl radical comprising, if appropriate, a hydroxyl functional group, or a (CH₂)ₘ—R₄ radical,

### R₂=COOR₅ or CN,

R₄=H or R₂, in the case where the starting material is a long-chain diester or dinitrile,
R₅=H, an alkyl radical having from 1 to 11 carbon atoms or also the residue of a diol, glycerol, a diglyceride or a triglyceride;
R₃=COOR₆, CN, H in the case of ethylene, CH₃ in the case of prop-1-ene or CH₂—CH₃ in the case of but-1-ene,
R₆=alkyl radical having from 1 to 4 carbon atoms,
m is a whole index within the range from 0 to 11,
n is a whole index within the range from 2 to 13,
R₂ is different from R₃.

The process according to the invention is characterized in that it comprises a stage in which two types of cross metathesis are alternated consecutively on an unsaturated compound I comprising at least one trivalent nitrile, acid or ester functional group.

A first type of cross metathesis (hereinafter “cm1”) is carried out with an acrylic compound II chosen from acrylonitrile, acrylic acid or an acrylic ester. In this type of cross metathesis cm1, one of the compounds I or II comprises a nitrile functional group and the other of these compounds comprises an acid or ester functional group.

Preferably, the metathesis reaction under consideration in this first cm1 type is a cross metathesis reaction between a monounsaturated acid or ester compound generally resulting from oleochemistry and acrylonitrile or a cross metathesis reaction between an unsaturated nitrile compound generally resulting from oleochemistry and an acid or acrylate acrylic compound and, in this case, preferably methyl acrylate.

The process had been developed in order to make use of starting materials resulting from renewable natural sources. However, it can also be applied to the analogous unsaturated compounds obtained by chemical synthesis.

This type of cm1 metathesis is preferably carried out in the presence of a metathesis catalyst of ruthenium carbene type, as described later.

A second type of cross metathesis (hereinafter “cm2”) is carried out with a light C₂ to C₄ alkene compound III, preferably with ethylene. In the present description of the invention, the synonymous terms “olefin” or “alkene” are used without distinction to denote a nonfunctionalized hydrocarbon comprising from 2 to 4 carbon atoms and a single covalent double bond between two of the carbon atoms.

The term “alternation” of these two types of metathesis in the process of the invention is understood to mean the sequence, at least once, of a cross metathesis of type 1 (cm1) and then of a cross metathesis of type 2 (cm2), according to the cm1-cm2 cycle; or, conversely, the sequence, at least once, of a metathesis of type 2 (cm2) and then of a metathesis of type 1 (cm1), i.e. the cm2-cm1 cycle; it being understood that the type of metathesis carried out first on the starting compound I can equally well be cm1 or cm2.

By way of example, the process according to the invention comprises one of the following cycles: cm1-cm2, cm2-cm1, cm1-cm2-cm1, cm2-cm1-cm2, cm1-cm2-cm1-cm2, cm2-cm1-cm2-cm1, cm1-cm2-cm1-cm2-cm1, cm2-cm1-cm2-cm1-cm2, and the like. The alternation of these two types of metathesis cm1 and cm2 can be carried out as many times as desired or as many times as necessary, according to the targeted improvement in nitrile-acid(ester) yield.

Mention may be made, as specific example, of the cycle [cm1 with acrylonitrile]-[cm2 with ethylene, “ethenolysis”]-[cm1 with acrylonitrile].

This alternation of cross metatheses according to the invention is also known hereinafter as “SWING cycle” or “SWING process”.

The term “consecutively” is understood to mean the sequence of the two types of metathesis which are defined above, without an intermediate separation stage other than the possible separation of light products with a carbon number of less than 15 and preferably of less than 5, such as light olefins, for example, or residual acrylic compounds (acrylonitrile or methyl acrylate, for example). For example, in the cm2-cm1-cm2 cycle, the second metathesis (of type 1) following upon a first metathesis (of type 2) is carried out directly on the products obtained under the provision of said first metathesis, and a third metathesis (of type 2) following upon the second metathesis is carried out directly on the products obtained on conclusion of this second metathesis.

It is this alternation of consecutive metatheses of type 1 and 2 according to the process of the invention which makes it possible to obtain an improved yield of ω-monounsaturated nitrile-ester (acid), compared with the yield obtained with just one metathesis of cm1 type or just one metathesis of cm2 type.

Starting compound I which can be used in the process of the invention is understood very particularly as an unsaturated fatty acid or ester, preferably of natural origin, of formula:

R₁—CH═CH—[(CH₂)_{q}—CH═CH]ₚ—(CH₂)ₙ—R₂ in which:
R₁ is H, an alkyl radical having from 1 to 11 carbon atoms, if appropriate comprising a hydroxyl functional group, or a (CH₂)ₘ—R₄ radical,
m is a whole index within the range from 0 to 11,
n is a whole index within the range from 2 to 13,
p is a whole index, p being equal to 0, 1 or 2,
q is an index equal to 0 or 1,
R₂ is COOR₅ or CN,
R₄ is H or R₂,
   R₅ is chosen from: H, an alkyl radical having from 1 to 11 carbon atoms or a radical comprising two or three carbon atoms carrying one or two hydroxyl functional group(s), or also the residue of a diglyceride or of a triglyceride in which each fatty acid of said glyceride residue is without distinction saturated or unsaturated.

The C═C double bonds of the compound I can have a cis or trans configuration.

The SWING cycle can be carried out both on ω-unsaturated fatty substances, the double bond of which is found at the chain end, and on fatty substances having an internal double bond, but ω-unsaturated fatty substances are preferably used.

According to an advantageous embodiment of the process of the invention, use is made of a starting compound I in which the index p is equal to 0 and R₁ is equal to H, that is to say a monounsaturated and ω-unsaturated compound of formula CH₂═CH—(CH₂)ₙ—R₂. Preferably in this case, the cycle of metatheses begins with a metathesis of cm1 type on the compound I, this being done in order to access bifunctional compounds. A nitrile-ester and a long-chain diester or dinitrile coproduct are thus formed. The following cross metathesis is a reaction involving an alkene (cm2), resulting in consumption of the long-chain diester or dinitrile, which is then partially converted into ω-unsaturated compound. A new cycle of cm1 type makes it possible to convert the ω-unsaturated compounds to nitrile-esters.

Preferably, the unsaturated compound I is an ω-unsaturated monounsaturated fatty acid or ester of formula CH₂═CH—(CH₂)ₙ—COOR₅.

Preferably, R₅ is H or an alkyl radical comprising from 1 to 11 carbon atoms, preferably from 2 to 11 carbon atoms.

According to a first advantageous embodiment of the process of the invention, ω-unsaturated fatty esters (acids), including when these ω-unsaturated fatty esters (acids) are present in the form of triglycerides, are employed with the acrylonitrile, during a first cross metathesis stage, in the presence of a first metathesis catalyst, the catalyst being added continuously. During this first stage, where the acrylonitrile reacts, ethylene is given off from the reaction and a mixture of nitrile-ester (nitrile-acid) and diester (diacid) is formed. On conclusion of this first stage, a second cross metathesis stage is carried out, this time using ethylene as reactant and using a second metathesis catalyst. On conclusion of this second cross metathesis stage, the proportion of diester (diacid) resulting from the homometathesis reaction of the first stage is significantly reduced, as it has been converted to ω-unsaturated fatty ester (acid)—starting reactant at the first stage.

In a third cross metathesis stage, the product mixture resulting from the second stage is again employed in the presence of acrylonitrile and of a third metathesis catalyst. The mixture obtained on conclusion of the third cross metathesis stage comprises a higher proportion of nitrile-ester (nitrile-acid) than the mixture resulting solely from the cross metathesis reaction involving acrylonitrile, with the same duration of contact with the acrylonitrile and involving the same amounts of catalyst.

According to another advantageous embodiment of the process of the invention, the unsaturated compound I is an ω-unsaturated monounsaturated fatty nitrile of formula CH₂═CH—(CH₂)ₙ—CN.

By way of example, ω-unsaturated fatty nitriles are employed in the process of the invention with acrylates, such as methyl acrylate, during a first cross metathesis stage, in the presence of a first metathesis catalyst preferably added continuously. During this first stage, where the acrylate reacts, ethylene is given off from the reaction and a mixture of nitrile-ester and dinitrile is formed. On conclusion of this first stage, a second cross metathesis stage is carried out, this time using ethylene as reactant and using a second metathesis catalyst. On conclusion of this second cross metathesis stage, the proportion of dinitrile resulting from the homometathesis reaction of the first stage is significantly reduced, as it has been converted to ω-unsaturated fatty nitrile—starting reactant in the first stage. In a third cross metathesis stage, the product mixture resulting from the second stage is again employed in the presence of acrylate and of a third metathesis catalyst. The mixture obtained at the conclusion of the third cross metathesis stage comprises a higher proportion of nitrile-ester than the mixture resulting solely from the cross metathesis reaction involving methyl acrylate, with the same duration of contact with the methyl acrylate and involving the same amounts of catalyst.

According to yet another advantageous embodiment of the process of the invention, use is made, as starting material, of a compound I in which the index p is other than 0 and/or R₁ is other than H. In this case, the cycle of metatheses preferably begins with a metathesis of cm2 type, this being done in order to access shorter olefins and in particular ω-unsaturated compounds. ω-Unsaturated compounds are preferred for metatheses of cm1 type. Moreover, the initial metathesis of cm2 type results in olefins which are more easily separated from the reaction medium, then composed of fatty nitriles or esters, due to their differences in physicochemical properties. By starting with a metathesis of cm2 type, it is therefore easy to retain, as a mixture, the unreacted heavy compounds, the products of the cross metathesis reaction (ω-unsaturated esters or nitriles) and the products resulting from the homometathesis reactions.

By way of example, use is made of unsaturated fatty acids, in the form of triglycerides or esters, with a light C₂ to C₄ alkene (ethylene, propylene, but-1-ene, but-2-ene) in a first cross metathesis stage of cm2 type (known as ethenolysis in the case of ethylene) carried out in the presence of a first metathesis catalyst, which is preferably added continuously. During this first stage, where the alkene reacts, alkenes are given off from the reaction and triglycerides comprising shorter unsaturated acids, including ω-unsaturated acids, are formed. On conclusion of this first stage, a second cross metathesis stage is carried out, this time using acrylonitrile as reactant (metathesis of cm1 type) and using a second metathesis catalyst. On conclusion of this second cross metathesis stage, the proportion of unsaturated ester resulting from the reaction of the first stage is significantly reduced, as it has been converted to unsaturated fatty nitrile-ester. Advantageously, the triglyceride obtained is subsequently hydrolyzed or alcoholyzed in order to release the glycerol and to recover fatty nitrile-acid or a fatty nitrile-ester.

This embodiment starting from triglycerides is, for example, carried out as follows:

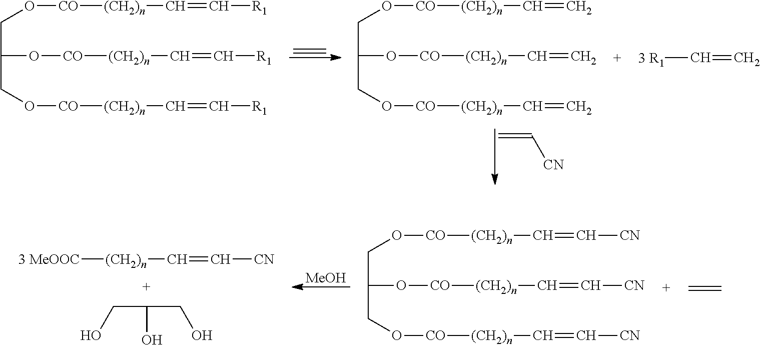

A second example of this cm2-cm1 embodiment is represented diagrammatically below:

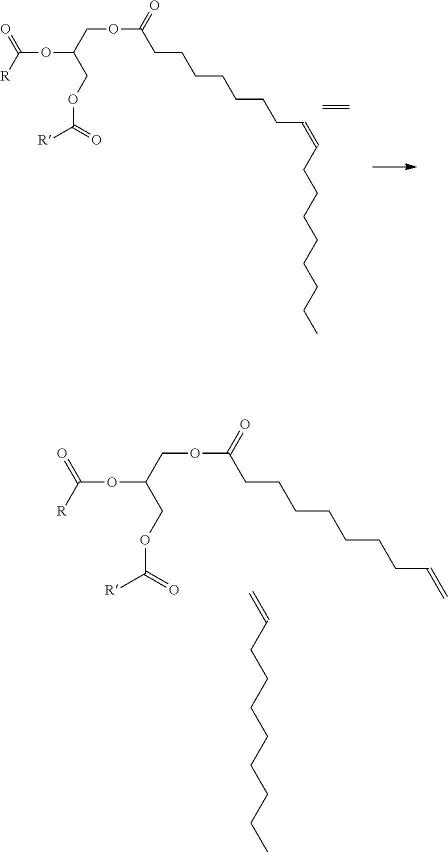

with release of 1-decene by distillation.

All the triglycerides are retained in the medium, as a mixture with the products of homometathesis between triglycerides.

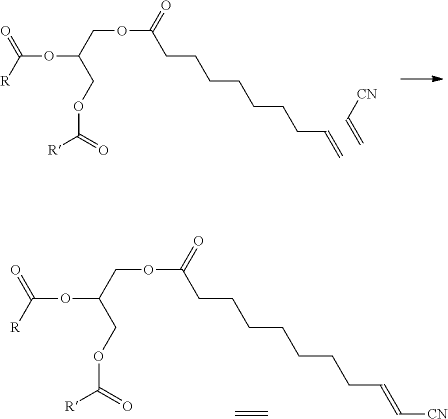

A new cm2 cross metathesis makes it possible to continue the conversion of the triglycerides and to convert the products of homometathesis of triglycerides.

The metathesis catalysts employed in the successive cm1 and cm2 stages can be identical or different. Preferably, different catalysts are used between the cross metathesis stages involving ethylene and those involving acrylates or acrylonitrile.

The reaction pressure and temperature for these two types of metathesis can be identical or different. Preferably, the reaction temperature is lower during cm2, in particular during the ethenolysis reaction, and the pressure is higher, in order to facilitate the dissolution of the alkene (in particular ethylene), than in the case of cross metathesis cm1 involving reactants of the acrylic type.

The cross metathesis reaction cm1 with a compound of acrylic type is carried out under conditions which are fully known. The metathesis reaction is preferably carried out at a reaction temperature between 20 and 120° C. and the pressure is between 1 and 30 bar in the presence of a ruthenium-based catalyst. It will preferably be carried out at low pressure between 1 and 10 bar and more preferably at atmospheric pressure when the cross metathesis results in the formation of a light compound, for example ethylene, in order to make possible easy release thereof. The reaction can be carried out without solvent or in the presence of solvent, such as, for example, tolulene, xylenes, dichloromethane, benzene, chlorobenzene, dimethyl carbonate, diethyl carbonate or their mixtures.

The cross metathesis reaction cm2, for example with ethylene, known as ethenolysis, is preferably carried out at a reaction temperature between 20 and 120° C. and the pressure is between 1 and 30 bar in the presence of a ruthenium-based catalyst. It will preferably be carried under a pressure between 1 and 20 bar and more preferably at a pressure greater than 5 bar in order to make possible easy dissolution of the ethylene in the organic phase. The reaction is carried out in the reaction medium, that is to say with solvents and catalyst residues, from the previous cross metathesis stage. Preferably, a purging stage in order to remove the residual acrylic compound precedes the ethenolysis stage. Likewise, a purging stage in order to remove the residual long alkenes is preferably carried out after a cross metathesis of cm2 type on an ester having an internal unsaturation.

The separation of the reaction products is said to be greatly facilitated as a result of the smaller amount of homometathesis products present in the reaction products. At the end of the swing cycle, the medium is composed mainly of the ester(or acid) nitrile, the starting ester(or acid) or nitrile and the homometathesis products. The products obtained can be separated by various techniques as a result of their significant differences in physicochemical properties: distillation at atmospheric pressure or under reduced pressure (significant difference in the boiling points and vapor pressures), crystallization (significant difference in the melting points), liquid-liquid extraction (significant difference in the solubilities of the products due to the difference introduced by the number of functional groups). Preferably, the separation technique selected will be distillation, as all the products can be distilled under reduced pressure.

The main advantage of the swing process of the invention is that it results directly:
in unsaturated nitrile-acids or nitrile-esters with selectivities of greater than 75%, preferably of greater than 80%, indeed even of greater than 90%;
the homometathesis products representing less than 25%, indeed even less than 10%, with respect to the total amount of products of the swing process.

Another subject matter of the invention is a process for the synthesis of a saturated α,ω-amino ester (or acid), preferably having a long chain, that is to say comprising from 6 to 17 carbon atoms, starting from an unsaturated compound I comprising at least one trivalent nitrile, acid or ester functional group, characterized in that it comprises:
a first stage of synthesis of a monounsaturated nitrile-ester (acid) by cross metatheses according to the swing cycle of the invention, in which two types of cross metathesis cm1 and cm2, as defined above, are alternated consecutively, starting with one or other of these types of metathesis; then
a stage of hydrogenation of the monounsaturated nitrile ester (acid) obtained in the preceding stage.

The α,ω-amino esters (acids) resulting from the process of the invention are manufactured from nitriles-esters (acids) devoid of homometathesis coproducts. They thus make possible the manufacture of polymers of better quality, with reduced production costs.

The formula of the nitrile-ester (acid) and thus that of the α,ω-amino ester (acid) synthesized depend essentially on that of the starting compound I.

In the case of a starting compound I resulting from oleochemistry, that is to say obtained from natural renewable fatty esters or acids, R₁ is either H or an alkyl radical or a functional alkyl radical comprising a trivalent (CN, COOH or COOR) functional group.

R₁ is H when the natural fatty ester is, for example, subjected to an ethenolysis or, in some cases, to a pyrolysis. The formula of the α,ω-amino ester/acid obtained is then directly related to the —(CH₂)ₙ— radical of the fatty ester. Thus it is that n is equal to 7 with oleic acid, to 4 with petroselenic acid, to 8 from ricinoleic acid (ester) subjected to a pyrolysis, to 10 from lesquerolic acid (ester) subjected to a pyrolysis, and the like, as described in the patent document FR2912741.

R₁ is an alkyl radical when, in (CH₂)ₘ—R₄, R₄ is H. This corresponds to the use in the process of a natural monounsaturated fatty acid, such as, for example, oleic acid, palmitoleic acid, petroselenic acid, lauroleic acid, and the like.

R₁ is a functional alkyl radical when, in (CH₂)ₘ—R₄, R₄ is a radical representing a trivalent CN, COOH or COOR functional group which is identical to R₂. The compound is then in the diacid, diester or dinitrile form. The formula of the compound then exhibits a symmetry. The preparation of compounds of this type, in particular by metathesis, is described in the abovementioned patent documents FR2912741, FR2938533 and FR2941694.

As regards the acrylic compound of cm1, the choice of the trivalent functional group R₃ is related to the nature of the trivalent functional group of the other compound, R₃ having to be nitrile when R₂ is ester/acid and, vice versa, ester/acid when R₂ is nitrile.

Advantageously, when the starting material is an oil, the first cross metathesis of cm2 type always results in a triglyceride (heavy compound) which is easily separated from the light olefins which are formed. A metathesis of cm1 type is then carried out with acrylonitrile, which gives compounds of triglyceride type carrying nitrile functional groups. After a low temperature hydrolysis or trans-esterification stage, glycerol, long-chain fatty esters or acids and short-chain nitriles-acids (or esters) are obtained, which it then becomes possible to separate. In comparison with a process in which ω-unsaturated esters or acids would have been isolated before carrying out the cross metathesis of cm1 type, there is a saving in number of separation stages and thus in energy consumption.

Advantageously, the process of the invention comprises a cm1 cross metathesis with acrylonitrile carried out on a compound I chosen from 9-decenoic acid or methyl 9-decenoate, resulting from the ethenolysis (or other cross metathesis with an alkene) of oleic acid or methyl oleate or also of a triglyceride, 10-undecenoic acid or methyl 10-undecenoate, resulting from the cracking of ricinoleic acid or methyl ricinoleate, oleic acid or methyl oleate, 9-octadecenedioic acid or methyl 9-octadecenedioate, resulting from the homometathesis or fermentation of oleic acid, erucic acid and methyl erucate, or 12-tridecenoic acid or methyl 12-tridecenoate, resulting from lesquerolic acid.

Advantagously, the process of the invention comprises a cm1 cross metathesis with the acrylic ester (acid) carried out on a compound I chosen from 9-decenenenitrile, resulting from 9-decenoic acid, 10-undecenenitrile, resulting from 10-undecenoic acid, 9-octadecenenitrile or oleonitrile, resulting from oleic acid, 9-octadecenedinitrile, resulting from 9-octadecenedioic acid, eruconitrile or 12-tridecenonitrile, resulting from lesquerolic acid.

The catalysis of the metathesis reaction has been the subject of a great many studies and the development of sophisticated catalytic systems. Mention may be made, for example, of the tungsten complexes developed by Schrock et al. (J. Am. Chem. Soc., 108 (1986), 2771) or Basset et al., Angew. Chem., Ed. Engl., 31 (1992), 628. “Grubbs” catalysts have more recently appeared (Grubbs et al., Angew. Chem., Ed. Engl., 34 (1995), 2039, and Organic Letters 1 (1999), 953), which are ruthenium-benzylidene complexes operating in homogeneous catalysis. Finally, studies have been carried out for the preparation of immobilized catalysts, that is to say, catalysts having an active principle which is that of the homogeneous catalyst, in particular ruthenium-carbene complexes, immobilized on an inactive support. The objective of these studies is to increase the selectivity of the cross metathesis reaction with regard to side reactions, such as “homometatheses” between the reactants brought together. They relate not only to the structure of the catalysts but also to the effect of the reaction medium and the additives which may be introduced.

The process according to the invention advantageously uses a metathesis catalyst of ruthenium-carbene type.

The ruthenium-carbene catalysts are preferably chosen from charged or uncharged catalysts of general formula:

(X1)a(X2)bRu(carbene C)(L1)c(L2)d(L3)e

in which:
a, b, c, d and e are identical or different integers, with a and b equal to 0, 1 or 2; c, d and e equal to 0, 1, 2, 3 or 4;
X1 and X2, which are identical or different, each represent a charged or uncharged and monochelating or polychelating ligand; mention may be made, by way of examples, of halides, sulfate, carbonate, carboxylates, alkoxides, phenolates, amides, tosylate, hexafluorophosphate, tetrafluoroborate, bis(triflyl)amide, an alkyl, tetraphenylborate and derivatives. X1 or X2 can be bonded to (L1 or L2) or to the (carbene C) so as to form a bidentate or chelate ligand on the ruthenium; and
L1, L2 and L3, which are identical or different, are electron-donating ligands, such as phosphine, phosphite, phosphonite, phosphinite, arsine, stilbene, an olefin or an aromatic compound, a carbonyl compound, an ether, an alcohol, an amine, a pyridine or derivative, an imine, a thioether or a heterocyclic carbene; L1, L2 or L3 can be bonded to the (carbene C) so as to form a bidentate or chelate ligand, or a tridentate ligand.
The (carbene C) is represented by the general formula: C_(R1)_(R2), for which R1 and R2 are identical or different groups, such as hydrogen or any other functionalized or nonfunctionalized hydrocarbon group of saturated, unsaturated, cyclic, aromatic, branched and/or linear type. Mention may be made, by way of examples, of ruthenium alkylidene, benzylidene, benzylidene ether or cumulene complexes, such as vinylidenes Ru═C═CHR or allenylidenes Ru═C═C═CR1R2 or indenylidenes.

A functional group (which makes it possible to improve the retention of the ruthenium complex in an ionic liquid) can be grafted to at least one of the ligands (X1, X2, L1 or L2 or to the carbene C. This functional group can be charged or uncharged, such as, preferably, an ester, an ether, a thiol, an acid, an alcohol, an amine, a nitrogenous heterocycle, a sulfonate, a carboxylate, a quaternary ammonium, a guanidinium, a quaternary phosphonium, a pyridinium, an imidazolium, a morpholinium or a sulfonium.

The metathesis catalyst can optionally be rendered heterogeneous on a support in order to facilitate the recovery/recycling thereof.

The cross metathesis catalysts of the process of the invention are preferably ruthenium carbenes described, for example, in Aldrichimica Acta, Vol. 40, No. 2, 2007, pp. 45-52.

The examples of such catalysts are the Grubbs catalysts, the Hoveyda-Grubbs catalysts, the Piers-Grubbs catalysts and other metathesis catalysts of the same type, whether “1st generation” or “2nd generation”.

The Grubbs catalysts are based on a ruthenium atom surrounded by 5 ligands:
2 anionic ligands, such as halides,
2 electron-donating ligands, such as trialkylphosphines or saturated N-heterocyclic carbenes (known as “NHC ligand”),
an alkylidene group, such as methylene “═CR2” groups, which are substituted or unsubstituted.

These metathesis catalysts are categorized into two categories, according to the nature of their electron-donating ligands L:
those which comprise 2 phosphine ligands (and not a saturated NHC ligand), which were developed first, are catalysts of 1st generation type,
those which comprise 1 saturated NHC (heterocyclic carbene) ligand are catalysts of 2nd generation type.

One type of “Hoveyda-Grubbs” catalyst comprises, among the electron-donating ligands, a benzylidene ether chelating ligand and either a phosphine (1st generation) or a saturated NHC ligand (2nd generation) generally substituted by two mesityl (Mes) groups.

Another type of “Piers-Grubbs” catalyst forms a cationic complex comprising 4 ligands which does not require the dissociation of a ligand before the reaction.

The 1st generation Grubbs catalysts prove to be particularly well suited to the cm2 metathesis according to the process of the invention. The 2nd generation Grubbs catalysts instead prove to be better suited to the cm1 metathesis. The 2nd generation Hoveyda-Grubbs catalysts are particularly well suited to cm1 metatheses with acrylonitrile.

Preferably, for the cm2 cross metathesis reactions with a light olefin, L1, L2 and L3 are not heterocyclic carbenes. The 1st generation catalysts are preferred in the case of metatheses of cm2 type.

Preferably, for the cm1 cross metathesis reactions involving an acrylic compound (acrylic acid, acrylonitrile or alkyl acrylate), L1, L2 or L3 is a heterocyclic carbene, preferably a saturated N-heterocyclic carbene. The 2nd generation catalysts are preferred in the case of metatheses of cm1 type.

Preferred catalysts for the cm1 cross metathesis stages involving an acrylic compound are, for example, the catalyst Umicore M51 (sold by Umicore) of formula (A) below, and the 2nd generation Hoveyda-Grubbs catalyst, also known as Hoveyda II (sold by Sigma-Aldrich, and Materia), of formula (B) below.

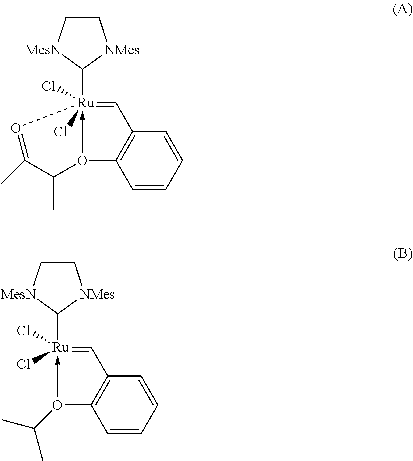

Other compounds sold by Omega Cat are also highly suitable (groups C and D below), or else those sold by Aldrich (E to J below), or also those sold by STREM and Umicore (M2, M3₁, M4₁ and M5₁ below).

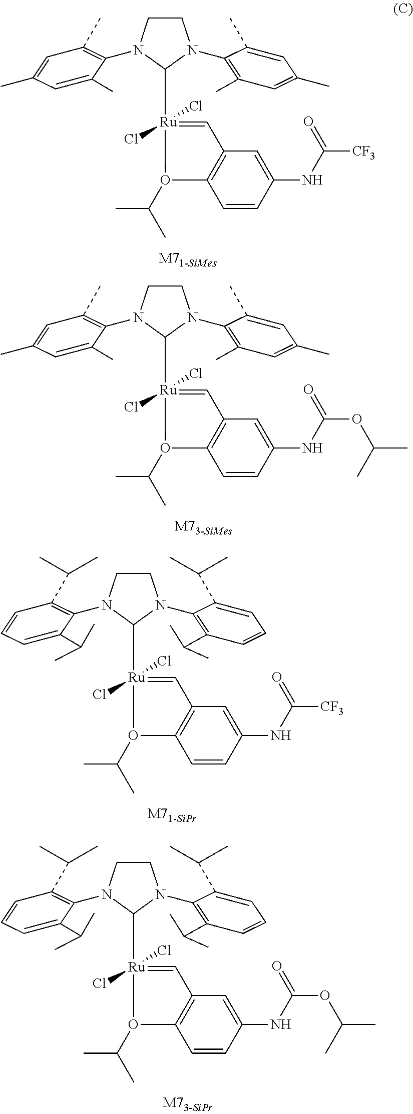

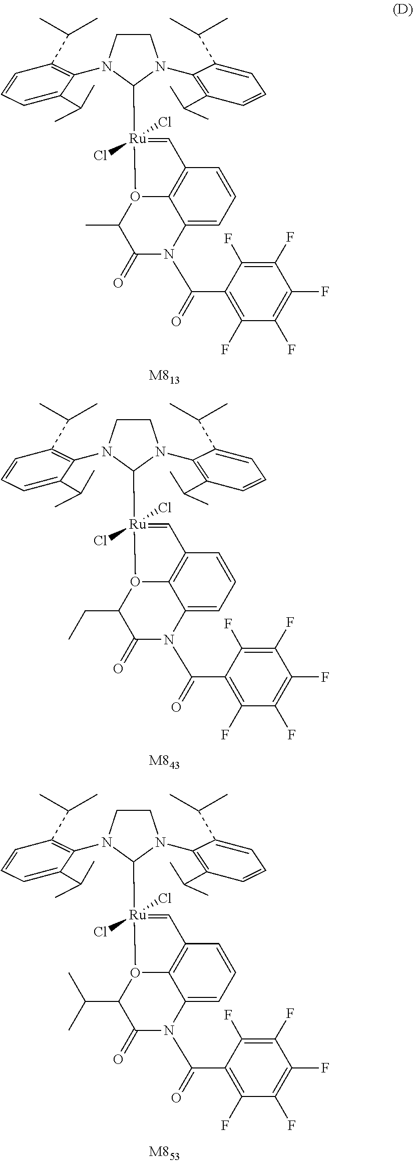

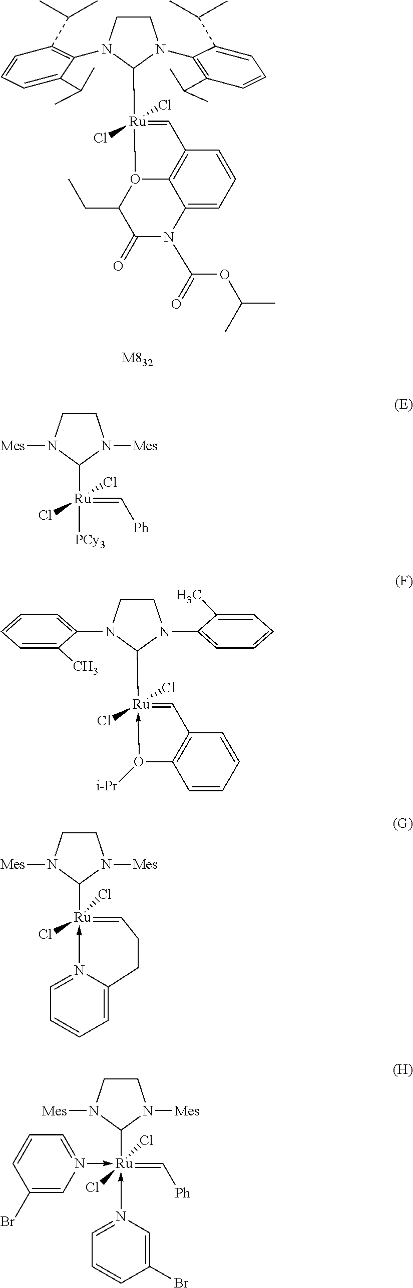

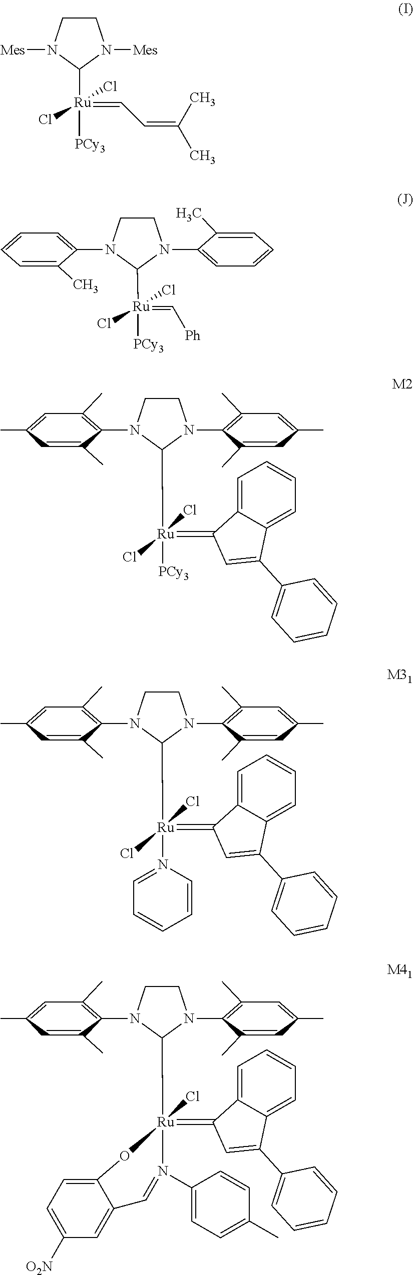

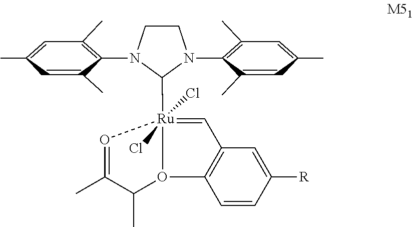

The catalysts for cm2 cross metathesis with light olefins, in particular the ethenolysis catalysts, of the process of the invention are preferably ruthenium carbenes described, for example, in Aldrichimica Acta, Vol. 40, No. 2, 2007, pp. 45-52, and in Chemfiles Vol. 9, No. 6. Preferred catalysts for the cm2 cross metathesis stages are in particular the M1 catalyst (sold by Umicore) and the 1st generation Grubbs catalysts (sold by Sigma-Aldrich and Materia) having the formulae (N, O and P) below.

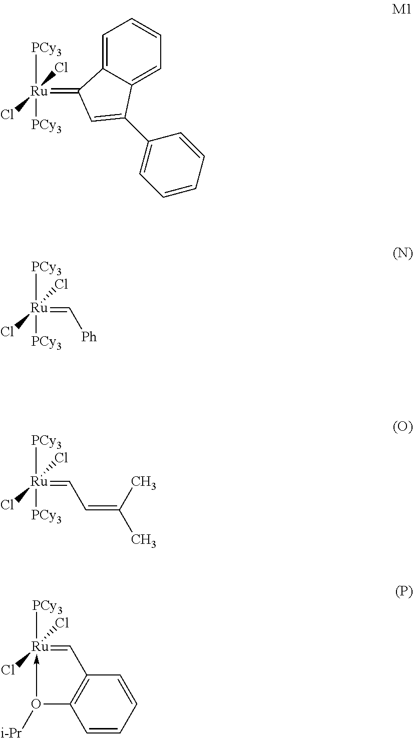

The amount of ruthenium metathesis catalyst introduced during the SWING cycle is chosen so that it ensures the completed possible conversion of the reactant (compound I) present in the charge. It is observed that said catalyst(s) is(are), under the operational conditions of the metathesis stage, transformed after the reaction; it is exhausted or deactivated and loses its catalytic activity in terms of metathesis—it will subsequently be denoted by the term of “degraded” for said reaction. In the batch process, the amount of the catalyst can easily be regulated in order to give the desired conversion at complete degradation of the catalyst. Advantageously, the amount of catalyst present during the metathesis reaction is characterized by a “turnover number” or “number of rotations”, equal to the [reactant which has reacted]/[catalyst] molar ratio, within the range from 5000 to 200 000 and preferably from 10 000 to 50 000.

The metathesis reaction time is chosen as a function of the reactants and operating conditions employed and in order to reach the end of the reaction.

As the metathesis is an equilibrium reaction, it is preferable to shift this equilibrium in order to proceed towards total conversion. In order to do this, in the case where the co-product of the reaction is a light olefin, such as ethylene, it is easy to “degas” the reactor from time to time or continuously in order to force the removal of the light products and thus to proceed towards total conversion. In the case where the co-product is a heavier olefin, optionally a functional one, the extraction operation is more problematic insofar as it is necessary to keep the two reactants and the catalyst in the reaction medium. Furthermore, if it is necessary to separate, at least in part, the unsaturated nitrile-ester (acid) by distillation and to remove the light products before a possible hydrogenation, it is preferable to carry out the operation so that the metathesis catalyst remains in the heavy fraction with the nitrile-ester (acid) in order to use it in its role of hydrogenation catalyst. In this operation, during the separation, the very heavy compounds are not removed from the medium, which compounds will thus be hydrogenated with the heavy fraction, their separation occurring during a subsequent purification of the final amino acid/ester.

The other way of shifting the equilibrium is to use an excess of reactant, typically, in cm1, an excess of acrylonitrile or alkyl acrylate (generally methyl acrylate) and, in cm2, an excess of light alkene. From a process viewpoint, each cross metathesis is carried out to completion with the exhausting of the metathesis catalyst; the excess acrylate, acrylonitrile or ethylene is distilled for recycling.

The process of the invention can be employed according to a batch or continuous mode.

According to the batch mode, for example employing first an acrylic compound, the process of the invention is carried out in consecutive reactors. The first cross metathesis reaction cm1 is carried out in a first reactor and, on completion of this reaction, the reactor is purged in order to remove the residual acrylic compound (methyl acrylate and acrylonitrile are heavier compounds than olefins but have boiling points low enough to envisage a distillation under partial vacuum at 100-120° C.) and the charge is transferred into a second reactor, which is pressurized with ethylene. After cm2 ethenolysis, the reactor, brought back to atmospheric pressure, is purged in order to remove the residual ethylene and then the charge is transferred into another reactor. A cm1 metathesis is then again carried out with an acrylic compound, preferably identical to that used during the first cm1, in order not to complicate the subsequent separations. This alternation of cm1 and cm2 according to the invention can thus be continued as many times as necessary, preferably until the content of diester or dinitrile is less than 25% by weight, preferably less than 10% by weight and more preferably still less than 5% by weight, with regard to the total weight of products in the reactor on conclusion of the final cross metathesis reaction according to the process.

According to an alternative embodiment, the process of the invention is carried out in continuous mode. In a reactor which is provided in the form of a column, the fatty ester or fatty nitrile charge is fed at the top and the cross metathesis catalyst is fed continuously slightly below the feed point of the fatty ester or nitrile and preferably in the top quarter of the reactive column. The acrylic compound (acrylonitrile, acrylic acid or acrylate, as the case may be) is fed in the upper half of the column and below the feed point of the catalyst. The latter, being a light compound, goes up the column whereas the fatty nitrile or ester, being a heavy compound, goes down the column. The cross metathesis reaction takes place at the point of injection and dispersion of the catalyst, concomitantly with the homometathesis reaction. The products of these reactions, being heavy compounds, go down the column. At the column top, ethylene produced by the cross metathesis and homometathesis reactions and the acrylic compound, having high vapor pressures, are recovered and directed to a separation column. The acrylic compound is reinjected into the reactive column, in the upper half, in the vicinity of the point of injection of the fresh charge. A portion of the ethylene produced is purged and another portion is compressed and optionally injected at the bottom of the reactive column in the vicinity of the point of injection of the fresh ethylene charge. The height of the column partially determines the pressure at the point of injection of the ethylene. A high column thus makes it possible to achieve high ethylene pressures. As ethylene is a light compound, it then goes up the reactive column. The ethenolysis catalyst is injected in the lower part of the reactive column, that is to say between the point of injection of the acrylic compound and the point of injection of the ethylene, and preferably in the bottom quarter of the column. The ethenolysis reaction is a rapid reaction which preferentially converts the diester and the dinitrile into ω-unsaturated fatty ester and nitrile respectively. The latter are lighter than the diester and dinitrile and partially go up the reactive column in order to again react with the acrylic compound.

The residence times of the reactants in the column are regulated by the feed and withdrawal flow rates. A mixture rich in nitrile-ester and also comprising unsaturated fatty nitrile or ester but also the unconverted diester or dinitrile is withdrawn at the bottom of the reactive column. The mixture is then sent to a separation unit where the unreacted unsaturated fatty nitrile or ester is separated and returned at the top of the reactive column. The nitrile-ester produced is subsequently separated from the dinitrile or diester, for example before or after a hydrogenation stage, in the case where the process of the invention additionally comprises a stage of hydrogenation of the monounsaturated nitrile-ester (acid) obtained to give α,ω-amino ester (acid).

According to an advantageous embodiment, the hydrogenation is carried out in the presence of a metathesis catalyst from the preceding stage, this catalyst operating as hydrogenation catalyst. After a SWING metathesis cycle (cm1,cm2), the reaction medium comprising the ruthenium is then directly subjected to a hydrogenation. This is because the ruthenium metathesis catalyst has degraded on conclusion of the metathesis stage but the metal is still present in the reaction medium in a form appropriate for the hydrogenation stage. In this way, the unsaturated nitrile-ester/acid compound present in the reaction medium is hydrogenated in the presence of the metal of the 1st stage catalyst in its hydrogenation role.

Typically, the hydrogenation is carried out under hydrogen pressure and in the presence of a base. The pressure is between 5 and 100 bar, preferably between 20 and 30 bar. The temperature is between 50 and 150° C., preferably between 80 and 100° C. The base can, for example, be sodium hydroxide, potassium hydroxide, potassium tert-butoxide or ammonia. The base is generally used at a content of 10 to 80 mol %, with respect to the unsaturated nitrile-ester substrate.

The hydrogenation reaction can be carried out with or without solvent. In the case of a reaction in a solvent medium, the preferred solvents used for the metathesis and hydrogenation stages are aromatic solvents, such as toluene or xylenes, a chlorinated solvent, such as dichloromethane or chlorobenzene, dimethyl carbonate, alcohols, acetic acid, and the like.

On conclusion of this hydrogenation stage carried out without a specific hydrogenation catalyst, the degree of conversion of the nitrile functional group to give a primary amine is particularly high, even without addition of NH₃, and also, of course, the reduction of the olefinic unsaturation, without the carboxyl functional group having been affected.

The degraded metathesis catalyst can optionally be employed with in addition a conventional hydrogenation catalyst for the hydrogenation stage. Mention may be made, among metals conventionally used for hydrogenation, of nickel, palladium, platinum, rhodium, ruthenium or iridium. Preferably, the degraded metathesis catalyst might be supplemented with Raney nickel, palladium- or ruthenium-on-charcoal, or ruthenium-on-alumina, -silica or -silicon carbide. Thus, in a specific embodiment, the hydrogenation reaction is carried out in the presence of the degraded metathesis catalyst resulting from the first stage supplemented by a conventional hydrogenation catalyst. It can also be carried out in the presence of a solid support (charcoal, SiC, and the like) in order to simplify its recovery.

The saturated amino acids or amino esters having a long chain comprising from 6 to 17 carbon atoms, preferably from 8 to 17 carbon atoms, obtained according to the process of the invention are advantageously used as monomers in the synthesis of polymers, in particular of polyamides. The synthesis of these polymers is facilitated by the absence of impurities (homometathesis coproducts) of the monomers manufactured according to the process of the invention.

A further subject-matter of the invention is polymers obtained by polymerization of the α,ω-amino esters (acids) synthesized according to the processes defined above.

### EXAMPLES

The following examples illustrate the invention without limiting it.

### COMPARATIVE EXAMPLE

### Not in Accordance with the Invention

5 g of 10-undecenenitrile (30 mmol), 5.2 g of methyl acrylate (60 mmol) and 50 g of toluene are charged to a 250 ml jacketed glass reactor equipped with a mechanical stirrer, a reflux condenser, a temperature probe and a nitrogen inlet. The reactor is purged with nitrogen and heated to 100° C. 0.55 mg of 2nd generation Hoveyda-Grubbs catalyst (Aldrich, 0.003 mol % with respect to the 10-undecenenitrile) dissolved in 5 g of toluene is added to the reactor over a period of 3 hours via a syringe and a syringe driver. The conversion of the 10-undecenenitrile, determined by GC, is 69%. The selectivity for unsaturated C₁₂ nitrile-ester (cross metathesis product) is 19%.

### Example 1

### In Accordance with the Invention

The 10-undecenenitrile is purified by passing over alumina. The toluene is purified by passing over a molecular sieve.

5 g of 10-undecenenitrile (30 mmol), 5.2 g of methyl acrylate (60 mmol) and 50 g of toluene are charged to a 250 ml jacketed glass reactor equipped with a mechanical stirrer, a reflux condenser, a temperature probe and a nitrogen inlet. The reactor is purged with nitrogen and heated to 100° C. 0.38 mg of 2nd generation Hoveyda-Grubbs catalyst (Aldrich, 0.002 mol % with respect to the 10-undecenenitrile) dissolved in 5 g of toluene is added to the reactor over a period of 3 hours via a syringe and a syringe driver. The ethylene produced is continuously removed.

At the end of the addition of the catalyst, after purging the residual methyl acrylate, the reaction mixture is transferred into a 300 ml autoclave. 12 mg of 1st generation Grubbs catalyst (0.05 mol % with respect to the 10-undecenenitrile) are added. The autoclave is purged with nitrogen and pressurized under 5 bar of ethylene. It is heated to 50° C. and reaction is allowed to take place for 30 minutes.

After cooling and decompressing, followed by purging with nitrogen, the reaction mixture is reintroduced into the atmospheric pressure assembly. 2.6 g of methyl acrylate (30 mmol) are added. The reactor is purged with nitrogen and heated to 100° C. and then 0.19 mg of 2nd generation Hoveyda-Grubbs catalyst (0.001 mol % with respect to the 10-undecenenitrile) dissolved in 5 g of toluene is added over a period of 1 hour. The conversion of the 10-undecenenitrile, determined by GC, is 90%. The selectivity for unsaturated C₁₂ nitrile-ester (cross metathesis product) is 76%.

These examples show that, for one and the same content of 2nd generation Hoveyda-Grubbs catalyst, a better conversion of the 10-undecenenitrile and a much higher selectivity for cross metathesis product are obtained with the process according to the invention (example 1).

### Hydrogenation Stage

The reaction mixture obtained in example 1 is transferred into a 300 ml autoclave. 0.5 g of Raney nickel, washed beforehand with methanol, is added. The autoclave is purged with nitrogen and then 5 bar of ammonia and 30 bar of hydrogen are introduced. The autoclave is heated to 100° C. and reaction is allowed to take place for 4 hours.

The conversion to unsaturated C₁₂ nitrile-ester, determined by gas chromatography, is greater than 99%. The selectivity for saturated C₁₂ amino ester is greater than 95%.

### Polymerization Stage

The product resulting from the hydrogenation is recovered by distillation under reduced pressure and is transferred into an autoclave. The autoclave is purged with nitrogen and then placed under a reduced pressure of 50 mbar. It is heated to 210° C. and reaction is allowed to take place for 2 hours. Polyamide 12 is thus obtained.

1. A process for the synthesis of a monounsaturated nitrile-ester (acid) by cross metathesis starting from an unsaturated compound I comprising at least one trivalent nitrile, acid or ester functional group, wherein the process comprises a stage in which two types of cross metathesis are alternated consecutively:
a first type of cross metathesis cm1 with an acrylic compound II chosen from acrylonitrile, acrylic acid or an acrylic ester, one of the compounds I or II comprising a nitrile functional group and the other of these compounds II or I comprising an acid or ester functional group, and
a second type of cross metathesis cm2 with a light C₂ to C₄ alkene compound III.

2. The process as claimed in claim 1, in which unsaturated compound I is of formula:
R₁—CH═CH—[(CH₂)_{q}—CH═CH]ₚ—(CH₂)ₙ—R₂ in which:
R₁ is H, an alkyl radical having from 1 to 11 carbon atoms, if appropriate comprising a hydroxyl functional group, or a (CH₂)ₘ—R₄ radical,
m is a whole index within the range from 0 to 11,
n is a whole index within the range from 2 to 13,
p is a whole index equal to 0, 1 or 2,
q is an index equal to 0 or 1,
R₂ is COOR₅ or CN,
R₄ is H or R₂,
R₅ is chosen from: H, an alkyl radical having from 1 to 11 carbon atoms, a radical comprising two or three carbon atoms carrying one or two hydroxyl functional group(s), or also the residue of a diglyceride or of a triglyceride in which each fatty acid of said glyceride residue is without distinction saturated or unsaturated.

3. The process as claimed in claim 2, in which the index p is equal to 0, R₁ is equal to H and the cycle of metatheses preferably begins with a cm1 metathesis on the compound I of formula:
CH₂═CH—(CH₂)ₙ—R₂

4. The process as claimed in claim 2, in which the unsaturated compound I is a fatty acid or ester of formula:
CH₂═CH—(CH₂)ₙ—COOR₅, in which
R₅ is H or an alkyl radical having from 1 to 11 carbon atoms.

5. The process as claimed in claim 2, in which the unsaturated compound I is a nitrile of formula:
CH₂═CH—(CH₂)ₙ—CN.

6. The process as claimed in claim 2, in which the index p is other than 0 and/or R₁ is other than H, and the cycle of metatheses preferably begins with a cm2 metathesis.

7. The process as claimed in claim 4, wherein the cm1 metathesis reaction is carried out with acrylonitrile and a compound I chosen from 9-decenoic acid, methyl 9-decenoate, 10-undecenoic acid, methyl 10-undecenoate, oleic acid, methyl oleate, 9-octadecenedioic acid, methyl 9-octadecenedioate, erucic acid, methyl erucate, 12-tridecenoic acid and methyl 12-tridecenoate.

8. The process as claimed in claim 5, wherein the cm1 metathesis reaction is carried out between an acrylic ester (acid) and a compound I chosen from 9-decenenitrile, 10-undecenenitrile, 9-octadecenenitrile, oleonitrile, 9-octadecenedinitrile, eruconitrile or 12-tridecenonitrile.

9. The process as claimed in claim 6, wherein the cm2 cross metathesis reaction is carried out between a light C₂ to C₄ alkene III and a compound I chosen from natural oils, triglycerides, polyunsaturated fatty esters and acids, and their mixtures.

10. The process as claimed in claim 1, wherein it comprises at least the sequence of stages cm1-cm2-cm1 or cm2-cm1-cm2.

11. The process as claimed in claim 1, wherein the compound I is a triglyceride and the process comprises at least the sequence of stages cm2-cm1, optionally followed by an alcoholysis or hydrolysis stage.

12. The process as claimed in claim 1, wherein the cross metathesis is carried out in the presence of a ruthenium catalyst chosen from charged or uncharged catalysts of general formula:
(X1)ₐ(X2)_{b}Ru(carbene C)(L1)_{c}(L2)_{d}(L3)ₑ,
in which:
a, b, c, d and e are identical or different integers, with a and b equal to 0, 1 or 2; c, d and e equal to 0, 1, 2, 3 or 4;
X1 and X2, which are identical or different, each represent a charged or uncharged and monochelating or polychelating ligand, X1 or X2 can be bonded to Y1 or Y2 (L1 or L2) or to the (carbene C) so as to form a bidentate or chelate ligand on the ruthenium; and
L1, L2 and L3, which are identical or different, are electron-donating ligands;
being possible for L1, L2 or L3 to be bonded to the (carbene C) so as to form a bidentate or chelate ligand, or a tridentate ligand, the (carbene C) being represented by the general formula: C_(R1)_(R2), for which R₁ and R₂ are identical or different groups.

13. The process as claimed in claim 12, wherein the amount of catalyst present during the metathesis reaction is characterized by a turnover number, equal to the [reactant which has reacted]/[catalyst] molar ratio, within the range from 5000 to 200 000.

14. The process as claimed in claim 12, wherein the cm1 metathesis reaction is carried out in the presence of a catalyst in which L1, L2 and/or L3 is a heterocyclic carbene.

15. The process as claimed in claim 12, wherein the cm2 metathesis reaction is carried out in the presence of a catalyst in which L1, L2 and L3 are not heterocyclic carbenes.

16. The process as claimed in claim 12, wherein the cm1 and/or cm2 cross metathesis is carried out in the presence of a ruthenium catalyst corresponding to one of the following formulae:

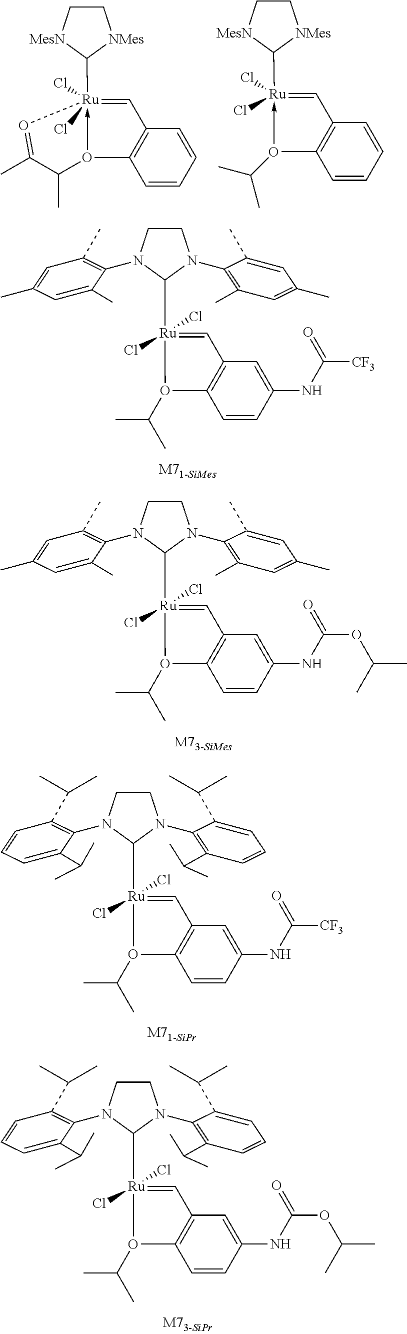

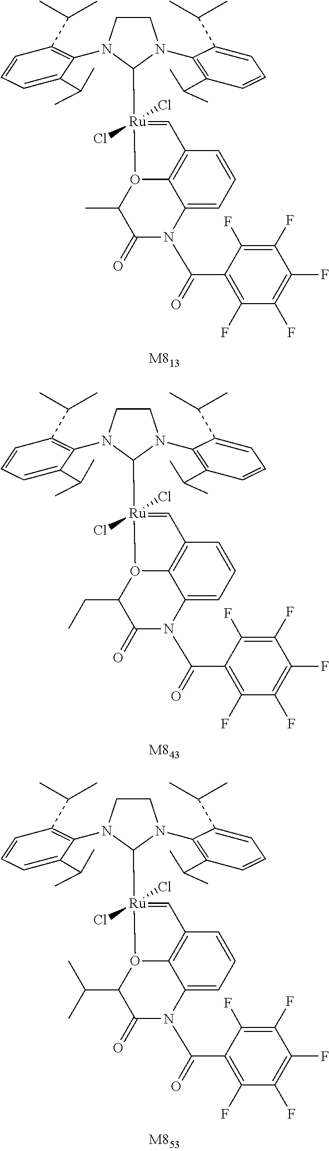

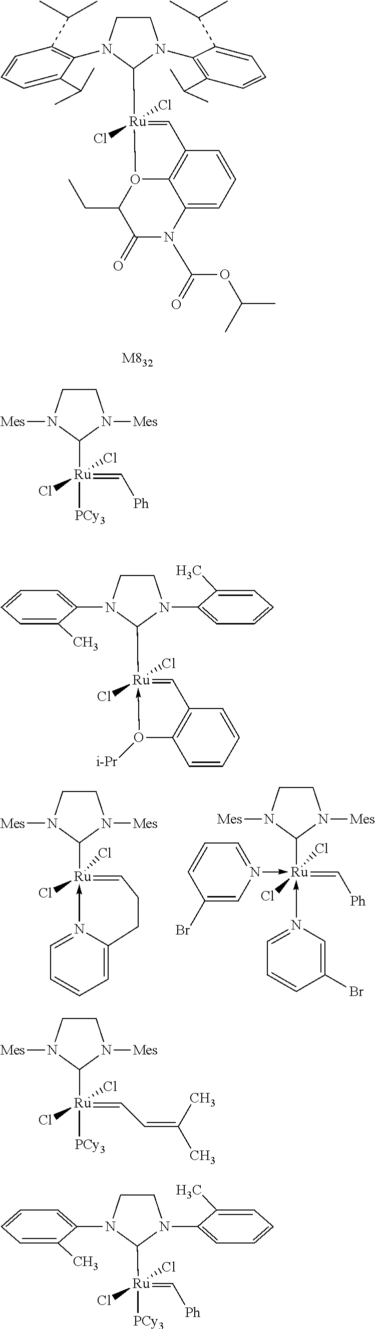

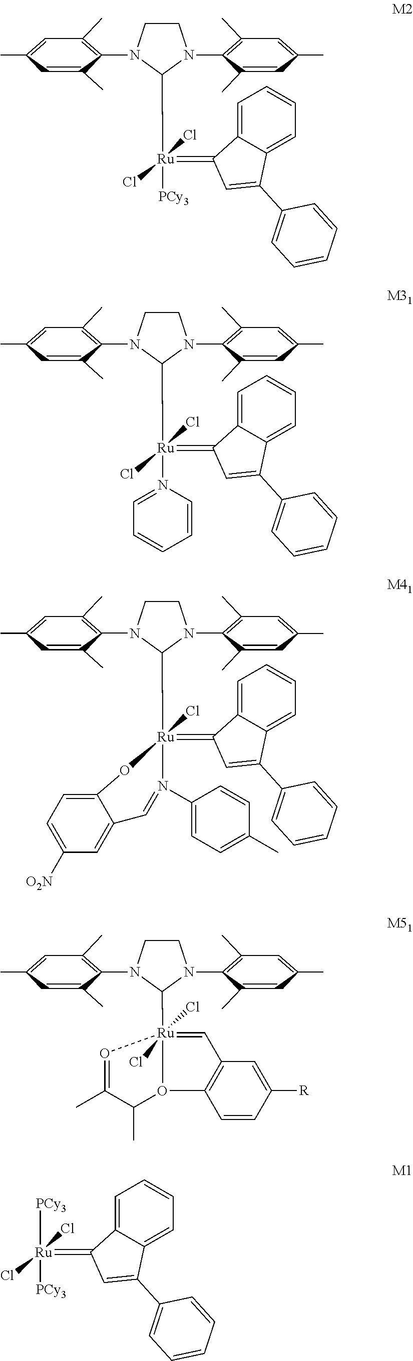

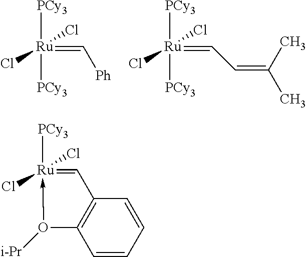

17. The process as claimed in claim 1, additionally comprising a stage of hydrogenation of the monounsaturated nitrile-ester (acid) obtained, so that a saturated α,ω-amino ester (acid) is obtained.

18. The process as claimed in claim 17, wherein the hydrogenation is carried out in the presence of a metathesis catalyst from the preceding stage, this catalyst operating as hydrogenation catalyst.

19. The process as claimed in claim 17, additionally comprising a stage of synthesis of polyamide by polymerization using the α,ω-amino ester (acid) obtained on conclusion of the hydrogenation stage.

20. A polymer, obtained by polymerization of α,ω-amino ester (acid) synthesized according to the process of claim 17.
